(19) United States
(12) Patent Application Publication
(10) Pub. No.: US 2014/0274770 A1
(43) Pub. Date: Sep. 18, 2014

(54) PCR ASSAYS AND REAGENTS FOR MOLECULAR DETECTION OF INFECTIOUS AGENTS

(71) Applicant: Quidel Corporation, San Diego, CA (US)

(72) Inventor: Todd Denison Pack, San Diego, CA (US)

(73) Assignee: Quidel Corporation, San Diego, CA (US)

(21) Appl. No.: 14/203,403

(22) Filed: Mar. 10, 2014

Related U.S. Application Data
(60) Provisional application No. 61/780,111, filed on Mar. 13, 2013.

(51) Int. Cl. C12Q 1/68

(52) U.S. Cl. CPC C12Q 1/689
USPC 506/9; 435/6.11; 506/16; 435/283.1

(57) ABSTRACT

The present disclosure is directed to PCR-based assays and compositions for use in molecular detection of viral, bacterial and parasitic infectious agents in body fluid or tissue samples, and in particular to multiplex assays, as well as to solid reagent compositions for use in such assays.

### CROSS-REFERENCE TO RELATED APPLICATIONS

This application claims the benefit of U.S. Provisional Application No. 61/780,111, filed Mar. 13, 2013, incorporated herein by reference in its entirety.

### TECHNICAL FIELD

The invention is directed to PCR-based assays for use in detecting pathogens in body fluid or tissue samples, in particular to multiplex assays, and to solid reagent compositions for use in such assays.

### BACKGROUND

PCR-based assays for detection of various pathogens, particularly viruses, bacteria and parasites, in clinical samples offer the advantages of high sensitivity and reproducibility, and can be carried out much more rapidly than traditional culturing methods, which can take multiple days. In many cases, rapid analysis is essential in order to properly treat infected individuals and, if necessary, implement procedures to prevent further transmission of infection. Pathogens of significance, which are discussed further below, include influenza A and B, human respiratory syncytial virus (RSV) A and B, human metapneumovirus (hMPV), herpes simplex virus 1 and 2 (HSV-1 and HSV-2), Varicella-zoster virus (VZV, also referred to as HSV-3), Clostridium difficile (C. duff), Staphylococcus aureus (SA), Staphylococcus epidermis (SE), Group B streptococcus, adenovirus, and parasites such as, for example, Cryptosporidium species, Entamoeba species including E. histolytica, Giardia lamblia, and Microsporidia.

In general, PCR-based molecular testing allows for sensitive detection of these and other pathogens in patient specimens, in less time than culture testing. However, most PCR protocols nonetheless employ multiple preparation steps, which can be time-consuming, and stringent precautions may be needed to avoid contamination of samples.

### SUMMARY

In one aspect, the invention provides a composition in solid form, comprising:

at least two PCR analyte primer pairs, each pair substantially complementary to a different target DNA sequence derived from a selected pathogen;

a PCR control primer pair substantially complementary to a process control DNA sequence;

at least one analyte probe for specific binding to each said target DNA sequence;

a control probe for binding to the process control DNA sequence;

a thermostable enzyme having DNA polymerase activity; and

deoxyribonucleotides dATP, dCTP, dGTP and dTTP or dUTP;

and wherein said analyte primer pairs and control primer pair are designed such that amplification and detection of said different target DNA sequences and said process control DNA sequence can be performed simultaneously using the same thermal cycling conditions on a thermocycler. For example, the melting temperatures (Tm) of primer/binding site duplexes for the different analytes, and for the process control, may be within 3° C., within 2° C., or within 1° C. or less, in the PCR reaction environment.

In some embodiments, the composition is in solid form, for example, in lyophilized form. In some embodiments, the composition is a lyophilized mixture.

In another embodiment, the solid composition is provided in combination with a rehydration solution that comprises manganese acetate. The manganese acetate in some embodiments is present at a concentration in the rehydration solution of between about 0.1-20 mM, 0.1-10 mM, 0.5-10 mM, 0.5-8 mM, 0.5-6 mM, 0.1-6 mM, 0.1-5 mM, 0.5-5 mM, 0.1-3 mM, or 0.5-3 mM.

In various embodiments, the different target DNA sequences are derived from at least two pathogens selected from influenza A, influenza B, Human respiratory syncytial virus (RSV) A, RSV B, and human metapneumovirus (hMPV); or from at least two pathogens selected from Herpes simplex virus 1, Herpes simplex virus 2, and Varicella-zoster virus (VZV).

In some embodiments, the process control DNA sequence is a cDNA for bacteriophage MS2.

Typically, the thermostable enzyme has reverse transcriptase activity; an example is a polymerase from Thermus aquaticus.

In some embodiments, the different target DNA sequences are transcribed from RNA of RSV and hMPV, respectively.

In some embodiments, the different target DNA sequences are transcribed from RNA of influenza A and influenza B, respectively.

In some embodiments, the different target DNA sequences are transcribed from RNA of HSV1, HSV2, and VZV (HSV3), respectively.

In some embodiments, the different target DNA sequences are transcribed from RNA of C. difficile toxin A and B, respectively.

In a similar aspect, a composition comprises at least two PCR analyte primer pairs, each pair substantially complementary to a different target DNA sequence derived from Clostridium difficile; a PCR control primer pair substantially complementary to a process control DNA sequence; at least one analyte probe for specific binding to each said target DNA sequence; a control probe for binding to the process control DNA sequence; a thermostable enzyme having DNA polymerase activity; and deoxyribonucleotides dATP, dCTP, dGTP and dTTP or dUTP. In some embodiments, the composition is in solid form, and the analyte primer pairs and control primer pair are designed such that amplification and detection of the different target DNA sequences and the process control DNA sequence can be performed simultaneously using the same thermal cycling conditions on a thermocycler.

In some embodiments, the melting temperatures (Tm) of primer/binding site duplexes for the different analytes and for the process control are within 3° C. in the PCR reaction environment. In some embodiments, the different target nucleic acid sequences are derived from tcdA and tcdB of Clostridium difficile. In a further embodiment, the different target DNA sequences are transcribed from RNA of tcdA and tcdB genes of Clostridium difficile, respectively.

In some embodiments, the process control DNA sequence is a cDNA for bacteriophage MS2. In a further embodiment, the thermostable enzyme has reverse transcriptase activity. In an additional embodiment, the enzyme is a polymerase from Thermus aquaticus.

In some embodiments, a method is provided for identifying the presence or absence of a Clostridium species in a sample, the method comprising:

a) spiking a sample suspected of containing the target nucleic acid sequence with a process control sequence, to form a spiked solution;

b) exposing the spiked solution to lysing conditions to form a lysed solution;

c) contacting with said lysed solution, to form a mixture, an amplification solution comprising
(i) at least two PCR analyte primer pairs, each pair specific for a different target nucleic acid sequence originating from a Clostridium species;
(ii) a PCR control primer pair specific for said process control sequence;
(iii) at least one analyte probe specific for each said different target nucleic acid sequence;
(iv) a control probe specific for said process control sequence;
(v) a thermostable enzyme having DNA polymerase activity; and
(vi) deoxyribonucleotides dATP, dCTP, dGTP and dTTP or dUTP;

d) producing an amplicon from at least one target nucleic acid sequence in the mixture, if present, using a single set of thermocycling conditions in a thermocycler; and

e) monitoring an analyte probe to determine the presence or absence of the target nucleic acid sequences.

In one embodiment, the amplification solution comprises manganese acetate. In one embodiment, the amplification solution comprises a concentration of manganese acetate that is between 0.1-20 mM or between 0.5-10 mM.

In some embodiments, the sample is a stool sample.

In some embodiments, the method further comprises prior to spiking the sample, processing the sample comprising (a) adding the sample to a first processing buffer to produce a buffered sample; and (b) adding a portion of the buffered sample to a second processing buffer.

In some embodiments, the at least two PCR analyte primer pairs are each specific for a different target nucleic acid sequence originating from Clostridium difficile.

In some embodiments, a kit is provided, wherein the kit comprises the composition comprising the at least two PCR analyte primer pairs substantially complementary to/specific for target nucleic acid sequences derived from Clostridium difficile; a PCR control primer pair substantially complementary to a process control DNA sequence; at least one analyte probe for specific binding to each said target DNA sequence; a control probe for binding to the process control DNA sequence; a thermostable enzyme having DNA polymerase activity; and deoxyribonucleotides dATP, dCTP, dGTP and dTTP or dUTP.

In some embodiments, the kit comprises a first container containing the aforesaid composition, and a second container containing a rehydration solution. In one embodiment, the rehydration solution comprises manganese acetate. The manganese acetate in some embodiments is present at a concentration in the rehydration solution of between about 0.1-20 mM, 0.1-10 mM, 0.5-10 mM, 0.5-8 mM, 0.5-6 mM, 0.1-6 mM, 0.1-5 mM, 0.5-5 mM, 0.1-3 mM, or 0.5-3 mM

In some embodiments, the kit comprises a third container containing a solution of MS-2 phage. In some embodiments, the kit comprises a fourth container containing a first process buffer, and a fifth container containing a second process buffer. In some embodiments, the rehydration solution comprises manganese acetate.

In some embodiments, a kit useful for carrying out multiplex PCR analysis is described, the kit comprising: a first container containing a composition as described above, in solid form; and a second container containing a rehydration solution. In some embodiments, the rehydration solution comprises manganese acetate, which can be at a concentration noted herein. In some embodiments, the solid composition may correspond to any of the selected embodiments described above. The kit may further contain a third container containing a solution of MS-2 phage (process control). The kit may also contain additional containers containing one or more process buffers. In some embodiments, the kit includes a fourth container containing a first process buffer and a fifth container containing a second process buffer. In some embodiments, the first process buffer comprises a sodium azide solution, NaOH, and lithium dodecyl sulfate. In another embodiment, the second process buffer comprises a sodium azide solution, NaCl, Tris, EDTA, and a control plasmid.

In a related aspect, the invention provides a method for identifying the presence or absence of at least two target nucleic acid sequences, each derived from a selected pathogen, in a sample, the method comprising:

a) spiking a sample suspected of containing the target nucleic acid sequences with a process control sequence, to form a spiked solution;

b) exposing the spiked solution to lysing conditions to form a lysed solution;

c) providing a solid composition comprising
(i) at least two PCR analyte primer pairs, each pair specific for a different nucleic acid sequence, which include said target nucleic acid sequences;
(ii) a PCR control primer pair specific for said process control sequence;
(iii) at least one analyte probe specific for each said different nucleic acid sequence;
(iv) a control probe specific for said process control sequence;
(v) a thermostable enzyme having DNA polymerase activity; and
(vi) deoxyribonucleotides dATP, dCTP, dGTP and dTTP or dUTP;

d) hydrating the solid composition with a solution to form a hydrated solution;

e) contacting the hydrated solution with the lysed solution of (b) to form a mixture;

f) producing an amplicon; e.g. by polymerase chain reaction (PCR), from the process control sequence and from said at least two target nucleic acid sequences in the mixture, if present, using a single set of thermocycling conditions in a thermocycler; and

g) monitoring the analyte probes, as provided in the composition of (c)(iii), to determine the presence or absence of the target nucleic acid sequences.

The method may further comprises one or more of the steps of:

after said exposing, contacting the lysed solution with a solid support having affinity for nucleic acids to form a nucleic acid bound support;

washing the nucleic acid bound support; and

exposing the nucleic acid bound support to conditions suitable to release nucleic acid from the solid support to form a released nucleic acid solution.

In some embodiments, the method is completed within 3 hours, and in some embodiments, within 2.5 hours.

In some embodiments, no separate nucleic acid extraction step is performed.

In some embodiments, the target nucleic acid sequences are RNA sequences, and the PCR is preceded by reverse transcription of the RNA sequences to cDNA sequences.

In some embodiments of the method, the pathogens (analytes), combinations of analytes, process control, and exemplary primers include those described for the solid compositions above.

In some embodiments of the method, useful for carrying out a respiratory panel, in which influenza A/B are assayed simultaneously, steps a)-e) are carried out, employing primer pairs specific for influenza A and for influenza B, respectively, to form a first mixture; steps a)-e) are carried out separately, employing primer pairs specific for RSV and hMPV, respectively, to form a second mixture; and steps f) and g) are then carried out simultaneously, with said first and second mixtures in separate vessels, under a single set of thermocycling conditions in a thermocycler.

In some embodiments, the method is useful for identifying the presence or absence of at least two target nucleic acid sequences, each derived from a selected pathogen, in a sample, comprises the steps of:

a) spiking a sample suspected of containing the target nucleic acid sequence with a process control sequence, to form a spiked solution;

b) exposing the spiked solution to lysing conditions to form a lysed solution;

c) contacting with said lysed solution, to form a mixture, a solution comprising
(i) at least two PCR analyte primer pairs, each pair specific for a different nucleic acid sequence, which include said target nucleic acid sequences;
(ii) a PCR control primer pair specific for said process control sequence;
(iii) at least one analyte probe specific for each said different nucleic acid sequence;
(iv) a control probe specific for said process control sequence;
(v) a thermostable enzyme having DNA polymerase activity; and
(vi) deoxyribonucleotides dATP, dCTP, dGTP and dTTP or dUTP;

d) producing an amplicon from said at least one target nucleic acid sequence in the mixture, if present, using a single set of thermocycling conditions in a thermocycler; and

e) monitoring the analyte probes, as provided in the composition of (c)(iii), to determine the presence or absence of the target nucleic acid sequences.

A similar method useful for identifying the presence or absence of at least two target nucleic acid sequences, each derived from a separate gene or region for the same pathogen, in a sample, comprises the steps of:

a) spiking a sample suspected of containing the target nucleic acid sequences with a process control sequence, to form a spiked solution;

b) exposing the spiked solution to lysing conditions to form a lysed solution;

c) providing a solid composition comprising
(i) at least two PCR analyte primer pairs, each pair specific for a different nucleic acid sequence, which include said target nucleic acid sequences;
(ii) a PCR control primer pair specific for said process control sequence;
(iii) at least one analyte probe specific for each said different nucleic acid sequence;
(iv) a control probe specific for said process control sequence;
(v) a thermostable enzyme having DNA polymerase activity; and
(vi) deoxyribonucleotides dATP, dCTP, dGTP and dTTP or dUTP;

d) hydrating the solid composition with a solution to form a hydrated solution;

e) contacting the hydrated solution with the lysed solution of (b) to form a mixture;

f) producing an amplicon from the process control sequence and from said at least two target nucleic acid sequences in the mixture, if present, using a single set of thermocycling conditions in a thermocycler; and

g) monitoring the analyte probes, as provided in the composition of (c)(iii), to determine the presence or absence of the target nucleic acid sequences. In one embodiment, the at least two target nucleic acid sequences are each derived form Clostridium difficile.

In some embodiments, the method further comprises:

prior to spiking the sample, processing the sample comprising

(a) adding the sample to a first processing buffer to produce a buffered sample; and

(b) adding a portion of the buffered sample to a second processing buffer.

In selected embodiments of these methods, the pathogens (analytes), combinations of analytes, process control, and exemplary primers include those described above.

Also provided herein are exemplary primer sequences useful in embodiments of the compositions, kits, and methods. These include those disclosed in the tables herein.

These and other objects and features of the invention will become more fully apparent from a review of the following detailed description of the invention.

### BRIEF DESCRIPTION OF THE FIGURES

FIG. 1 shows an exemplary process for identifying the presence and/or absence of one or more target nucleic acid sequences.

### DETAILED DESCRIPTION

### I. Definitions

The terms below, as used herein, have the stated meanings unless indicated otherwise. Terms and abbreviations not defined should be accorded their ordinary meaning as used in the art. Note also that singular articles, such as “a” and “an”, encompass the plural, unless otherwise specified or apparent from context.

When a range of values is provided, it is understood that each intervening value, to the tenth of the unit of the lower limit unless the context clearly dictates otherwise, between the upper and lower limits of that range is also specifically disclosed. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm. Each smaller range between any stated or intervening value in a stated range and any other stated or intervening value in that stated range is encompassed by the disclosure. The upper and lower limits of the smaller ranges may be independently included or excluded in the range, and each range where either, neither or both limits are included in the smaller ranges is also encompassed by the disclosure, subject to any specifically excluded limit in the stated range. Where the stated range includes one or both of the limits, ranges excluding either or both of those included limits are also included.

By “specific to” (or “specific for”) a particular pathogen, with respect to PCR primers, is meant that the primers are substantially complementary, and in some embodiments exactly complementary, to selected primer binding sites in highly conserved regions of the genome of the target pathogen, or, in RT-PCR, to selected primer binding sites in cDNA transcribed from these regions. The sequences of the highly conserved regions may be consensus sequences from sequence alignment of multiple strains of the pathogen. The definition also applies to PCR probes.

By “substantially complementary”, with respect to a PCR primer or probe, is meant that the oligomer is sufficiently complementary to its binding site for efficient binding and amplification to proceed under the conditions of a PCR assay. In some embodiments, the oligomer is exactly complementary to its binding site, or to a consensus sequence for the binding site. However, there may be one or more mismatches between the primer and/or probe and the binding site in the analyte that are tolerated and still result in specific amplification and detection.

“Detection” of a target nucleic acid or analyte refers to determining the presence or the absence of the nucleic acid or analyte in a sample, where absence refers to a zero level or an undetectable level.

### II. PCR-Based Detection Method

Disclosed herein is a multiplex real time PCR-based assay for the qualitative differential detection and identification of multiple nucleic acid targets; e.g. influenza A and/or influenza B, or RSV and/or hMPV, or multiple targets from a single organism such as C. difficile, from patient test samples. The in vitro diagnostic test is directed towards the diagnosis of viral, bacterial and/or parasitic infections in patients, particularly human patients. In some embodiments, the assay provides differential detection of the presence or absence of multiple pathogens in a single assay. In another embodiment, the assay provides differential detection of the presence or absence of a pathogen by detecting multiple targets to the pathogen in a single assay. Advantageously, the PCR-based assays disclosed herein can be performed in less than 3 hours, and in some cases less than 2.5 hours.

In one aspect of the invention, the majority of the reagents employed in the assays are provided in solid form, for example in lyophilized form, and, in some embodiments, in a single container. After sample preparation, the solid mix of reagents is simply rehydrated and combined with the liquid sample. The assay can thus be carried out with a minimal amount of transfer of reagent solutions, greatly reducing the possibility of contamination or loss of sample, as well as the time needed for completion of the assay.

The components of the solid composition, described in more detail below, include:

(i) one or more PCR analyte primer pairs specific for a nucleic acid sequence derived from a pathogen to be detected (analyte), wherein each pair, when more than one is employed, is specific for a different nucleic acid sequence;

(ii) a PCR control primer pair specific for a process control sequence;

(iii) at least one analyte probe specific for each said different nucleic acid sequence;

(iv) a control probe specific for the process control sequence;

(v) a thermostable enzyme having DNA polymerase activity; and

(vi) deoxyribonucleotides dATP, dCTP, dGTP and dTTP/dUTP.

In some embodiments, the solid composition includes at least two PCR analyte primer pairs, each specific for a different nucleic acid sequence derived from a pathogen to be detected (analyte). More specifically, the primer pairs are specific for selected primer binding sites, which are typically in highly conserved regions of the genome of the pathogen to be detected, or, in RT-PCR, to selected primer binding sites in cDNA transcribed from these regions.

As described further below, the primers are designed, and thermocycling conditions selected, such that efficient amplification and detection of multiple target sequences can be performed simultaneously using the same thermal cycling conditions on a thermocycler. In some embodiments, the primers are designed such that the annealing/melting temperatures of primer/binding site duplexes for the different analytes, and for the process control, are approximately equivalent, e.g. within 3° C., within 2° C., or within 1° C. or less, in the PCR reaction environment. In other embodiments, the annealing/melting temperatures of primer/binding site duplexes for the different analytes, and for the process control, are within 5° C., or are nearly equivalent, e.g. within 0.5° C. or less.

For example, an assay could include two targets, such as influenza A and B, or RSV and hMPV. Using primers such as those disclosed in the tables herein, these four analytes could be run simultaneously as a respiratory panel, since the primers are designed to be effective under the same thermal cycling conditions. Typically, the influenza A/B and RSV/hMPV assays are run in separate wells, albeit under the same cycling conditions. As another example, the assay may include two targets from the same pathogen, such as targets to toxin A and toxin B of C. difficile. Typically, the toxin A and toxin B assays are run in separate wells, albeit under the same cycling conditions.

Also contained in the solid composition are labeled probes corresponding to the (multiple) target species and to the process control, as well as reagents conventionally employed for PCR; e.g. a thermostable enzyme having DNA polymerase activity, and deoxyribonucleotides dATP, dCTP, dGTP and dTTP/dUTP. In one embodiment, the enzyme is a polymerase from Thermus aquaticus. The solid composition also typically includes one or more stabilizers.

Commonly assayed pathogens include, as described further below, respiratory viruses such as influenza A and B, human respiratory syncytial virus (RSV) A and B, and human metapneumovirus (hMPV); Herpes simplex virus 1 and 2 (HSV-1 and HSV-2), Varicella zoster virus (VZV), also known as Human herpes virus 3 (HHV-3), Clostridium difficile (C. diff), Group B Streptococcus (GBS), adenovirus, various Staphylococcus species, such as methicillin-resistant Staphylococcus aureus (MRSA), methicillin-sensitive Staphylococcus aureus (MSSA), methicillin-resistant coagulase-negative staphylococci (MRCNS), methicillin-sensitive coagulase-negative staphylococci (MSCNS), methicillin-resistant Staphylococcus epidermidis (MRSE) and methicillin-sensitive Staphylococcus epidermidis (MRSE), and parasites such as, for example, Cryptosporidium species, Entamoeba species including E. histolytica, Giardia lamblia, and Microsporidia. The assay may be used for detection of the presence or absence of these species, using, in selected embodiments, the primer and probe sequences disclosed herein. In general, these and other species may be assayed alone or in combination, in accordance with the invention, using specific primers and probes specific for regions of the genome that are highly conserved among different strains of the given pathogen.

The test sample may be any body fluid or tissue sample suspected of containing a target pathogen, collected according to procedures known in the art. For example, respiratory viruses may be detected in a nasal swab, nasophyrangeal swab, or nasal aspirate/wash specimens. As another example, the target pathogen(s) may be detected from a stool sample.

Extraction of nucleic acids from the test sample may be performed manually or automatically, as known in the art, using the appropriate reagents and following the manufacturer's instructions for automated systems. Automated sample extraction platforms include, for example, the nucliSENS® easyMAG® system (BioMérieux) or the MagNA Pure Compact system (Roche Diagnostics). In some embodiments, no extraction step is needed or performed.

A process control is added to an aliquot of every specimen prior to the extraction procedure. The process control serves to assure adequate nucleic acid extraction and to reflect the presence of any inhibitors that may be present in the sample. In some embodiments, the process control is stabilized MS2 bacteriophage.

In performing the assay, the solid reagent composition is rehydrated, in some embodiments using a manganese acetate-containing solution, and aliquots are placed in PCR reaction tubes or plate wells. Aliquots of prepared sample fluid, containing nucleic acids and process control, are then added. (Alternatively, the rehydrated reagents can be added to the fluid sample.) Amplification by PCR or RT-PCR is then carried out in a thermal cycling apparatus, such as the Life Technologies 7500 FastDx or Cepheid SmartCycler® II.

As noted above, the primers are designed such that efficient amplification and detection of multiple target sequences can be performed simultaneously, using the same thermal cycling conditions. Exemplary primer sets having this property are described below. Accordingly, a multiplex PCR or RT-PCR reaction can be carried out under optimized conditions in a single vessel, or in multiple vessels but under the same thermocycling conditions, generating amplicons for each of the target pathogens present in a sample.

For detection of viral pathogens, the amplification reaction can be an RT-PCR reaction, employing an enzyme with reverse transcriptase, DNA polymerase, and 5′-3′ exonuclease activities, e.g. a polymerase from Thermus aquaticus.

The labeled probes may be designed such that, during DNA amplification, the 5′ exonuclease activity of the polymerase enzyme cleaves the probe bound to the complementary DNA sequence, separating the quencher dye from the reporter dye on the probe, and thereby generating an increase in detectable fluorescent signal. With each amplification cycle, additional dye molecules are separated from their quenchers, resulting in additional signal. If sufficient fluorescence is achieved by a predetermined number of cycles, e.g. 40 cycles, the sample is reported as positive for the detected nucleic acid.

FIG. 1 depicts a non-limiting and exemplary method including sample processing and detection. A sample, such as a stool sample for detecting C. difficile, is obtained or supplied by a patient. A portion of the sample is added to a first process buffer (PB1). In the depicted embodiment, a supplied sampling swab suitable for obtaining a sample from a stool sample is used to collect a sample that is then added to the PB1. The swab is agitated, twirled or swirled in the PB1 for a period of time sufficient to transfer a suitable portion of the sample into the PB1. In one non-limiting embodiment, the sampling swab is swirled in PB1 for at least about 2-10 seconds (including at least about 2 seconds, 3 seconds, 4 seconds, 5 seconds, 10 seconds, etc.) to release a portion of the sample into the PB1. A portion of PB1 with added sample is added to a second process buffer (PB2) and mixed by a suitable method as known in the art. For example, about 20-50 μL (including at least about 20 μL, 25 μL, 30 μL, 35 μL, 40 μL, 45 μL, 50 μL etc.) of the PB1 with sample is added to PB2 and the PB1 with sample plus PB2 mixture is mixed by pipetting a portion (for example, 0.1-1.0 mL) of the mixture up and down several times, resulting in a diluted sample. Separately, a rehydration buffer is added to a suitable solid reagent composition as further described herein. A suitable amount of the rehydrated reagent composition is added to a suitable container for each assay. In an exemplary embodiment where the assay is a PCR molecular assay, the rehydrated reagent is added to each reaction tube or well on a well-plate. A suitable amount of the diluted sample is added to each sample assay well. The well plate may then be centrifuged or spun briefly, and is then inserted into a thermocycler such as a real-time PCR thermocycler for initiation of amplification.

### III. Assay Reagent Compositions and Kits

In one aspect of the invention, as noted above, the majority of the reagents employed in the multiplex PCR-based assay are provided in solid form, for example, as a lyophilized mixture. The composition includes, in solid form:

one or more PCR analyte primer pairs specific for a nucleic acid sequence derived from a pathogen to be detected, wherein each pair, when more than one is employed, is specific for a different nucleic acid sequence;

(ii) a PCR control primer pair specific for a process control sequence;

(iii) at least one analyte probe specific for each said different nucleic acid sequence;

(iv) a control probe specific for the process control sequence;

(v) a thermostable enzyme having DNA polymerase activity; and

(vi) deoxyribonucleotides dATP, dCTP, dGTP and dTTP/dUTP.

In some embodiments, the solid composition includes at least two PCR analyte primer pairs, each specific for a different nucleic acid sequence derived from a pathogen to be detected. Primers included together in a solid composition (also referred to as a PCR “master mix”) have sequences such that efficient amplification and detection of their respective target sequences can be performed simultaneously using the same thermal cycling conditions on a thermocycler.

The primer analyte pairs are preferably specific to highly conserved regions in the genome of the target pathogen(s). In one embodiment of the method, which employs RT-PCR, the primers are substantially complementary to selected regions of the cDNA generated by reverse transcription of highly conserved regions of RNA, such as viral, bacterial or parasite RNA. The sequences of the highly conserved regions used for primer design may be consensus sequences derived from multiple strains and/or subtypes of a pathogen.

In selected embodiments, the analyte primers include pairs of primers specific for selected regions of the genomes of (at least) two pathogens selected from the group consisting of influenza A, influenza B, Human respiratory syncytial virus (RSV) type A and/or B, human metapneumovirus (hMPV), Herpes simplex virus 1, Herpes simplex virus 2, Varicella-zoster virus (VZV), Clostridium difficile, Group B Streptococcus (GBS), adenovirus, methicillin-resistant Staphylococcus aureus (MRSA), methicillin-sensitive Staphylococcus aureus (MSSA), methicillin-resistant coagulase-negative staphylococci (MRCNS), methicillin-sensitive coagulase-negative staphylococci (MSCNS), methicillin-resistant Staphylococcus epidermidis (MRSE) and methicillin-sensitive Staphylococcus epidermidis (MRSE), and parasites such as, for example, Cryptosporidium species, Entamoeba species including E. histolytica, Giardia lamblia, and Microsporidia. In another embodiment, the analyte primer pairs include pairs of primers specific for selected and different regions of the genome of a pathogen such as C. difficile.

In one embodiment, for example, for use in detection of influenza A and/or B, the composition includes two pairs of analyte primers, complementary to cDNA sequences transcribed from RNA of influenza A and influenza B, respectively. In another embodiment, for use in detection of RSV and/or hMPV, the composition includes pairs of analyte primers complementary to cDNA sequences transcribed from RNA of RSV and hMPV, respectively. In a further embodiment, for use in detection of C. difficile, the composition includes pairs of analyte primers complementary to cDNA sequences transcribed from toxin A and toxin B of C. difficile.

As noted above, the composition also includes a further pair of primers, termed control primers, which are specific for a process control sequence present in the prepared sample. In one embodiment, the process control is bacteriophage MS2, such that the process control DNA sequence is a cDNA for bacteriophage MS2. The primers may be specific for a highly conserved region of the MS2 genome, e.g. the coat protein.

The control primer sequences are also selected such that efficient amplification and detection of the control sequence can be performed using the same thermal cycling conditions as used for the target analyte(s). In some embodiments, the control primers are designed such that the annealing/melting temperatures of primer/binding site duplexes are approximately equivalent to those of the analyte primer/binding site duplexes, e.g. within 3° C., within 2° C., or within 1° C. or less, in the PCR reaction environment.

Also contained in the solid composition are labeled probes corresponding to the (multiple) target species and to the process control. The probes are designed to bind to the target region to be amplified at a location between the two primer binding sites. Each probe can be labeled with a fluorophore at one terminus, e.g. the 5′ terminus, and a quencher at the other terminus, such that fluorescent resonance energy transfer (FRET) from the reporter is quenched by the quencher when the probe is intact and is activated when the probe is cleaved. An example is a Taqman® probe, which is cleaved by the 5′ exonuclease activity of the polymerase enzyme during amplification.

Accordingly, an exemplary composition, for use in detection of influenza A and/or B, includes one or more analyte probes for specific binding to an influenza A cDNA sequence and one or more analyte probes for specific binding to an influenza B cDNA sequence. An exemplary composition for use in detection of RSV and/or hMPV includes one or more analyte probes for specific binding to an RSV cDNA sequence and one or more analyte probes for specific binding to an hMPV cDNA sequence. An exemplary composition for use in detection of C. difficile includes one or more analyte probes for specific binding to a first C. difficile cDNA sequence, such as from toxin A, and one or more analyte probes for specific binding to a second C. difficile cDNA sequence, such as from toxin B. In all cases, one or more process control probes is also included.

Also contained in the solid composition are reagents conventionally employed for PCR; e.g. a thermostable enzyme having DNA polymerase activity, and deoxyribonucleotides dATP, dCTP, dGTP and dTTP/dUTP. In one embodiment, for use in RT-PCR, the thermostable enzyme also has reverse transcriptase activity. Typically, the enzyme is a polymerase from Thermus aquaticus. The solid composition typically includes one or more stabilizers.

In some embodiments, a method is provided for identifying the presence or absence of a Clostridium species in a sample, the method comprising:

a) spiking a sample suspected of containing the target nucleic acid sequence with a process control sequence, to form a spiked solution;

b) exposing the spiked solution to lysing conditions to form a lysed solution;

c) contacting with said lysed solution, to form a mixture, a solution comprising
(i) at least two PCR analyte primer pairs, each pair specific for a different target nucleic acid sequence originating from a Clostridium species;
(ii) a PCR control primer pair specific for said process control sequence;
(iii) at least one analyte probe specific for each said different target nucleic acid sequence;
(iv) a control probe specific for said process control sequence;
(v) a thermostable enzyme having DNA polymerase activity; and
(vi) deoxyribonucleotides dATP, dCTP, dGTP and dTTP or dUTP;

d) producing an amplicon from said at least one target nucleic acid sequence in the mixture, if present, using a single set of thermocycling conditions in a thermocycler; and

e) monitoring the analyte probes, as provided in the composition of (c)(iii), to determine the presence or absence of the target nucleic acid sequences.

In some embodiments, the method further comprises:

prior to spiking the sample, processing the sample comprising

(a) adding the sample to a first processing buffer to produce a buffered sample; and

(b) adding a portion of the buffered sample to a second processing buffer.

In some embodiments, the at least two PCR analyte primer pairs are each specific for a different target nucleic acid sequence originating from Clostridium difficile.

In some embodiments, a kit is provided, wherein the kit comprises the composition comprising the at least two PCR analyte primer pairs substantially complementary to/specific for target nucleic acid sequences derived from Clostridium difficile; a PCR control primer pair substantially complementary to a process control DNA sequence; at least one analyte probe for specific binding to each said target DNA sequence; a control probe for binding to the process control DNA sequence; a thermostable enzyme having DNA polymerase activity; and deoxyribonucleotides dATP, dCTP, dGTP and dTTP or dUTP.

In some embodiments, the kit comprises a first container containing the aforesaid composition, and a second container containing a rehydration solution. In some embodiments, the kit comprises a third container containing a solution of MS-2 phage. In some embodiments, the kit comprises a fourth container containing a first process buffer, and a fifth container containing a second process buffer. In some embodiments, the rehydration solution comprises manganese acetate.

Also provided are kits in a single container, containing components of an exemplary solid reagent composition as described above, e.g., one ore more, preferably at least two, PCR analyte primer pairs, each pair substantially complementary to a different target DNA sequence derived from a selected pathogen; a PCR control primer pair substantially complementary to an process control DNA sequence; at least one analyte probe for specific binding to each said target DNA sequence; a control probe for binding to the process control DNA sequence; a thermostable enzyme having DNA polymerase activity, and preferably having reverse transcriptase activity; deoxyribonucleotides dATP, dCTP, dGTP and dTTP/dUTP; and, in some embodiments, one or more stabilizers. These components are provided as a solid composition in a first container in the kit.

A second container within the kit contains a rehydration solution, that in one embodiment contains manganese acetate, for use in rehydrating the solid composition. In one embodiment, the manganese acetate is at a concentration in the rehydration solution of between about 0.1-20 mM, 0.1-10 mM, 0.5-10 mM, 0.5-8 mM, 0.5-6 mM, 0.1-6 mM, 0.1-5 mM, 0.5-5 mM, 0.1-3 mM, or 0.5-3 mM. In another embodiment, the concentration of manganese acetate in the final assay is between about 0.1-20 mM, 0.1-10 mM, 0.5-10 mM, 0.5-8 mM, 0.5-6 mM, 0.1-6 mM, 0.1-5 mM, 0.5-5 mM, 0.1-3 mM, or 0.5-3 mM. In some embodiments, the kit contains a third container containing a solution of the process control, which may be MS-2 bacteriophage. External process controls for the pathogens being assayed may also be included. The kit will also contain instructions for using these components in carrying out PCR-based assays. In some embodiments, the kit includes software-driven assay protocols for use in commercial PCR instrumentation (such as the Life Technologies 7500 FastDx or Cepheid SmartCycler® II), which may be provided on a CD.

In some embodiments, the kit comprises a sample processing kit and a PCR kit as described above. The sample processing kit may comprise one or more containers comprising process buffer(s) for processing the sample. The PCR kit preferably comprises a container comprising a solid reagent composition, and second container comprising a rehydration solution, that in one embodiment comprises manganese acetate, for use in rehydrating the solid composition. Other components as described above may also be included such as, but not limited to, a third container containing a process control solution and instructions.

In some embodiments, the solid composition may correspond to any of the selected embodiments described above. The kit may also contain additional containers containing one or more process buffers. In some embodiments, the kit includes a fourth container containing a first process buffer and a fifth container containing a second process buffer. In some embodiments, the first process buffer comprises a sodium azide solution, NaOH, and lithium dodecyl sulfate. In another embodiment, the second process buffer comprises a sodium azide solution, NaCl, Tris, EDTA, and a control plasmid.

In one specific embodiment, the sample processing kit comprises a first and a second processing buffer, a solid reagent composition, and a rehydrating solution, each provided in a separate container. In an exemplary embodiment, the first processing buffer comprises 0.001-0.5 mL/mL of a sodium azide solution, 0.001-0.5 mL/mL sodium hydroxide, 0.001-0.5 mL/mL lithium dodecyl sulfate, and water qs. In non-limiting embodiments, the first processing buffer comprises 0.001-0.01 mL/mL, 0.004-0.01 mL/mL, 0.005-0.01 mL/mL, 0.001-0.05 mL/mL of a sodium azide Solution; 0.001-0.5 mL/mL, 0.001-0.05 mL/mL, 0.02-0.05 mL/mL, 0.01-0.05 mL/mL sodium hydroxide; 0.0001-0.5 mL/mL, 0.0001-0.005 mL/mL, 0.001-0.005 mL/mL lithium dodecyl sulfate; and water qs. In one non-limiting embodiment, the first processing buffer comprises 0.004 mL/mL of a 5% sodium azide solution, 0.014 mL/mL of 10N sodium hydroxide, 0.0014 g/mL lithium dodecyl sulfate, and 0.9876 mL/mL MG water. In an exemplary embodiment, the second processing buffer comprises a sodium azide solution, NaCl, Tris-HCl, EDTA, a control plasmid, and water qs. In an exemplary embodiment, the second processing buffer comprises 0.001-0.5 mL/mL of a sodium azide solution, 0.001-0.5 mL/mL NaCl, 0.001-0.5 Tris, 0.0001-0.5 mL/mL EDTA, and water qs. In non-limiting embodiments, the second processing buffer comprises 0.001-0.005 mL/mL, 0.0001-0.005 mL/mL, 0.001-0.05 mL/mL of a sodium Azide solution; 0.001-0.01 mL/mL, 0.001-0.05 mL/mL, 0.0001-0.05 mL/mL NaCl; 0.001-0.01 mL/mL, 0.005-0.1 mL/mL, 0.005-0.05 mL/mL Tris, and 0.0001-0.0005 mL/mL, 0.0001-0.001 mL/mL, 0.0001-0.0002 mL/mL, 0.0002-0.001 mL/mL EDTA, and water qs. In one non-limiting embodiment, the second processing buffer comprises 0.004 mL/mL of a 5% sodium azide solution, 0.0061 mL/mL of 5M NaCl, 0.0100 mL/mL of 1M Tris-HCl, 0.0002 mL/mL of 0.5 EDTA, a control plasmid (0.0000005 mL/mL), and 0.97965 mL/mL MG water.

### IV. Exemplary Analytes

The current PCR-based assays are useful for detection of various pathogens, particularly viruses, bacteria and parasites, in clinical samples. Pathogens of significance include, for example, influenza A and B, human respiratory syncytial virus (RSV) A and B, human metapneumovirus (hMPV), herpes simplex virus 1 and 2 (HSV-1 and HSV-2) and Varicella-zoster virus (VZV, also referred to as HSV-3), Clostridium difficile (C. cliff), adenovirus, various Staphylococcus species, such as methicillin-resistant Staphylococcus aureus (MRSA), methicillin-sensitive Staphylococcus aureus (MSSA), methicillin-resistant coagulase-negative staphylococci (MRCNS), methicillin-sensitive coagulase-negative staphylococci (MRCNS), methicillin-resistant Staphylococcus epidermidis (MRSE) and methicillin-sensitive Staphylococcus epidermidis (MRSE), Group B streptococcus, Bordetella pertussis, Bordetella parapertussis, Bordetella holmesii, and parainfluenza 1-4. and parasites such as, for example, Cryptosporidium species, Entamoeba species including E. histolytica, Giardia lamblia, and Microsporidia.

MRSA is a strain of Staphylococcus aureus (S. aureus) bacteria, a common type of bacteria that may live on the skin and in the nasal passages of healthy people. MRSA has become one of the most dangerous infectious agents in the U.S. and elsewhere, with a higher mortality rate than HIV-AIDS. MRSA does not respond to some of the antibiotics generally used to treat staphylococcus and other bacterial infections.

Healthcare-associated MRSA (HA-MRSA) infections occur in people who are or have recently been in a hospital or other health-care facility. Many people may be at risk of MRSA infection due to receiving healthcare services in an environment where the MRSA bacteria are colonized on surfaces, healthcare workers, the patient or other patients. Community-associated MRSA (CA-MRSA) infections occur in otherwise healthy people who have not recently been in the hospital. In fact, MRSA has become a primary cause of skin and soft tissue infections among persons without extensive exposure to healthcare settings, and the outbreaks have occurred in athletic team facilities, correctional facilities, and military basic training camps.

In addition to methicillin-sensitive S. aureus (MSSA) and methicillin-resistant S. aureus (MRSA) strains, there are CNS, or CoNS, (coagulase-negative staphylococci) species, close relatives of the bacterium Staphylococcus aureus, commonly found in humans. Many strains of CNS are also resistant to methicillin (MRCNS) containing a similar SCCmec gene cassette mechanism as MRSA. Specifically, methicillin-resistant S. epidermidis (MRSE) is the species in the CNS complex of species most commonly seen among MRCNS carriers. Among immunocompromised patients, MRCNS, especially MRSE, can lead to infections and is a common cause of wound, blood and respiratory infections. MRSE can cause severe infections in immune-suppressed patients and those with central venous catheters.

Interventions for MRSA colonization through decolonization, isolation procedures, or restrictions in occupational activities among clinicians and patients would be more effective if there was a way to rapidly identify patients among healthcare workers who are colonized with MRSA. However, current identification systems are based on outdated, cumbersome, and time consuming technologies, such as culturing, and are focused only on MRSA. Therefore, the present disclosure meets a need for technologies that enable positive identification and differentiation of MRSA, MSSA, MRCNS and MSCNS using more rapid and informative tests with a high level of accuracy for both screening for colonization and diagnosis of infections. Exemplary methods, kits, primers and probes are disclosed in U.S. Patent Publication 2011/0312504 (U.S. Ser. No. 13/051,755), which is incorporated by reference herein, in its entirety.

Respiratory infections cause significant morbidity and mortality in both developed and developing countries. Influenza A and B, which are RNA viruses of the family Orhtomyxoviridae, infect an estimated 120 million people in the US, Europe and Japan, and cause the deaths of more than 250,000 people worldwide, each year. Pandemics of Influenza A occur about every 10 to 30 years, and epidemics of either Influenza A or B occur annually. The Centers for Disease Control (CDC) and the World Health Organization (WHO) maintain surveillance of influenza strains and make predictions of suitable strains for vaccine production.

Human respiratory syncytial virus (RSV) is a negative single-stranded RNA virus of the family Paramyxoviridae. RSV is the major cause of lower respiratory tract infection and hospital visits during infancy and childhood. In the United States, nearly all children will have been infected with the virus by 2-3 years of age. Of those infected with RSV, 2-3% will develop bronchiolitis, necessitating hospitalization. Two RSV subtypes, A and B, have been identified, with studies generally finding that RSV-A is responsible for the larger number of outbreaks and the more severe symptoms (Papadopoulos et al., 2004, Resp. Med. 98:879-882).

Human metapneumovirus (hMPV) is a negative single-stranded RNA virus of the family Paramyxoviridae, and may be the second most common cause (after RSV) of lower respiratory infection in young children. The virus appears to be distributed worldwide and to have a seasonal distribution, with its incidence comparable to that for the influenza viruses during winter. Serologic studies have shown that by the age of five, virtually all children have been exposed to hMPV, and re-infections appear to be common. hMPV generally causes mild respiratory tract infection; however, small children, the elderly and immunocompromised individuals are at risk for severe disease and hospitalization. Co-infection with RSV can occur, and is generally associated with more severe disease.

Sequence analyses of the hMPV genome have shown that hMPV strains can be divided into two main genetic lineages (A and B) representing two serotypes, each comprising two sublineages (A1, A2, B1, and B2) (B G van den Hoogen et al., 2001, Nat. Med. 7:719-24; 2004, Emerg. Infect. Dis. 10(4):658-66).

Herpes simplex virus 1 and 2 (HSV-1 and HSV-2) are DNA viruses of the family Herpesviridae. HSV-1 and HSV-2 are genetically and antigenically distinct forms of HSV. The consequences of HSV infection can range from inconsequential (cold sores) to highly morbid and fatal (neonates and immunocompromised). They can be a result of the primary infection by the virus or from a reactivation of the latent virus, causing recurrent episodes of the disease.

Varicella-zoster virus (VZV), also known as Human herpes virus 3 (HHV-3), is a DNA virus of the family Herpesviridae. Primary VZV infection results in chickenpox (varicella), which may result in complications including encephalitis or pneumonia. Even when clinical symptoms have resolved, VZV remains dormant in the nervous system of the infected person. In 10-20% of cases VZV reactivates, producing shingles. Serious complications include post herpetic neuralgia, zoster multiplex, myelitis, herpes ophthalmicus, or zoster sine herpete.

Because HSV-1, HSV-2, and VZV (HSV-3) all present with lesions that can be phenotypically difficult to differentiate, it is advantageous to have a sensitive and specific molecular assay to distinguish them.

Clostridium difficile (C. diff) is a gram positive, anaerobic, spore-forming bacillus that produces two major toxins, toxin A and toxin B, resulting in C. difficile associated disease (CDAD), which generates severe diarrhea and may lead to complications such as toxic megacolon and death. Traditional methods currently employed to diagnose CDAD include cytotoxic cell culture, lateral flow assays, and enzyme immunoassays; however, the sensitivity of these tests remains quite low, rendering them less useful diagnostically. Exemplary methods, kits and oligonucleotides are disclosed in U.S. Patent Publication 2010/0233717 (U.S. Ser. No. 12/741,147), and PCT Publication WO 2010/116290, each of which is incorporated by reference herein, in its entirety.

Adenoviruses are medium-sized (90-100 nm), nonenveloped icosahedral viruses (lacking an outer lipid bilayer) composed of a nucleocapsid and a double-stranded linear DNA genome. There are 57 described serotypes in humans, which are responsible for 5-10% of upper respiratory infections in children, and many infections in adults as well.

Staphylococcus aureus (SA) is responsible for approximately 25% of all bloodstream infections; amongst those, 26% to 47% are caused by methicillin-resistant strains (MRSA). The resulting bacteremia has a mortality rate of 25%-35%; thus the timely identification of SA and MRSA is necessary in order to provide effective antibiotic therapy. Current traditional methods for identification of SA and MRSA include culture and agglutination testing, followed by oxacillin susceptibility testing, which takes between 16 to 48 hours in order to obtain results. Current PCR-based methods require expensive instrumentation and must be performed in a highly complex molecular lab, rather than in a microbiology laboratory, a resource that many small to medium hospitals do not have access to.

Parasitic diseases caused by helminths and protozoa are major causes of human disease and misery in most countries of the tropics. They plague billions of people and kill millions annually, inflicting debilitating injuries such as blindness and disfiguration on additional millions. The World Health Organization estimates that one person in every four harbors parasitic worms. Parasitic worms and/or protozoans may be identified using the compositions and methods of the instant disclosure. For example, methods for detecting Acanthamoeba species, Anisakis species, Ascaris lumbricoides, Botfly, Balantidium coli, Bedbugs, Cestoidea (tapeworms), Chiggers, Cochliomyia hominivorax, Cryptosporidium species, Entamoeba species including E. histolytica, Fasciola hepatica and other liver flukes, Giardia species (e.g., G. lamblia), Hookworm, Leishmania, Linguatula serrata, Loa loa, Microsporidia, Paragonimus, Plasmodium falciparum, Schistosoma, Strongyloides stercoralis and other pinworms, mites, Toxoplasma gondii, Trypanosoma, Whipworm and Wuchereria bancrofti are provided.

### V. Exemplary Primers and Probes

As noted above, the primer pairs employed for production of amplicons from the target nucleic acids may be specific to selected highly conserved regions in the genomes of the target pathogen(s). By “specific to” (or “specific for”) is meant that the primers are substantially complementary, and in some embodiments exactly complementary, to selected primer binding sites in said highly conserved regions, or, in RT-PCR, to selected primer binding sites in cDNA transcribed from these regions. For the purpose of primer design, the primer binding sites are based on consensus sequences derived from alignment of these highly conserved regions from different strains of the target pathogens.

As described further below, the primers are designed such that efficient amplification and detection of multiple target sequences can be performed simultaneously, using the same thermal cycling conditions. Accordingly, the primers are designed such that the annealing/melting temperatures of the primer/binding site duplexes for the different analytes, and for the process control, are approximately equivalent, e.g. within 3° C., within 2° C., or within 1° C. or less, in the PCR reaction environment.

In some embodiments, the primer sets for different analytes are also designed such that there is no detectable cross-reaction among analytes, or with other common pathogens.

The above design parameters are particularly desirable for analytes which may be frequently analyzed together; for example, influenza A and influenza B, or, in a respiratory panel, influenza A and B, RSV, and hMPV. Another common combination includes HSV 1, HSV 2 and VZV (HSV 3). Other possible combinations include, for example, combinations of Staphylococcus aureus (SA) species, selected from MRSA, MSSA, MRCNS, MSCNS, MRSE and MSSE; combinations of Bordetella pertussis, Bordetella parapertussis, and Bordetella holmesii; and parainfluenza 1-4, and combinations of C. difficile toxin A and toxin B. In each case, a process control may be included. Generally, the number of analytes that can be detected in a single assay is limited by instrument capability and/or the number of available distinguishable labels. A typical number is two or three analytes plus a process control.

In one embodiment, an assay could include two targets, such as influenza A and B, or RSV and hMPV. In each of these assays, the process control MS2 bacteriophage coat protein and the two respective primers may be included.

In a further embodiment, these four analytes (influenza A/B, RSV, and hMPV) could be run simultaneously as a respiratory panel, since the primers are designed to be effective under the same thermal cycling conditions. In some embodiments, the influenza A/B and RSV/hMPV assays are run in separate wells, albeit under the same cycling conditions.

Similarly, the primers for HSV1, HSV2, and VZV (HSV3) are designed to be effective under the same thermal cycling conditions, in combination with process control primers.

### EXAMPLES

### Exemplary Assay Procedure

Sample Collection:

Nasal swabs, nasopharyngeal swabs, nasal aspirate/wash specimens, and stool specimens are obtained using standard techniques from symptomatic patients. The specimens are transported, stored, and processed according to established laboratory procedures.

Sample Preparation:

The process control is added to each aliquot of every specimen prior to the extraction procedure. The control serves to monitor inhibitors in the specimen, assures that adequate amplification has taken place and that nucleic acid extraction was sufficient.

Nucleic acids may be extracted from the specimens using, for example, the NucliSENS easyMAG System, following the manufacturer's instructions and using the appropriate reagents.

Rehydration of Master Mix:

The lyophilized master mix (solid reagent composition) is rehydrated using 135 μL of rehydration solution (manganese acetate solution). The master mix contains oligonucleotide primers and fluorophore and quencher-labeled probes targeting highly conserved regions of the target pathogens, e.g. viruses, as well as the process control sequence. The primers are complementary to highly specific and conserved regions in the genome of these viruses. The probes are dual labeled with a reporter dye attached to the 5′ end and a quencher attached to the 3′ end. This quantity of rehydrated master mix is sufficient for eight reactions.

Nucleic Acid Amplification and Detection:

15 μL of the rehydrated master mix is added to each reaction tube or plate well. 5 μL of nucleic acids (specimen with process control) is then added to the plate well or appropriately labeled tube. The plate or tube is then placed into a thermal cycling instrument, such as the Life Technologies 7500 FastDx or Cepheid SmartCycler II instrument.

Once the reaction tube or plate is added to the instrument, a software-driven assay protocol, typically provided with the kit components, is initiated. This protocol initiates reverse transcription of the viral RNA targets and process control, generating complementary DNA, and the subsequent amplification of the target amplicons. The assay is typically based on Taqman® chemistry and uses an enzyme with reverse transcriptase, DNA polymerase, and 5′-3′ exonuclease activities. During DNA amplification, this enzyme cleaves the probe bound to the complementary DNA sequence, separating the quencher dye from the reporter dye. This step generates an increase in fluorescent signal upon excitation by a light source of the appropriate wavelength. With each cycle, additional dye molecules are separated from their quenchers resulting in an increase in the fluorescent signal. If sufficient fluorescence is achieved within a given number of cycles, the sample is reported as positive for the detected nucleic acid.

### Example 1

### Clostridium difficile Assay

A multiplex real-time TaqMan Assay® was developed to detect and differentiate toxin A and toxin B of Clostridium difficile. The assay master mix (solid composition) contained primers/probes for detection and differentiation of these two analytes, as shown in Table 1 above.

83 stool samples were collected and placed in a sample container. Sample was collected from the sample container using a swab and inserted into a separate container comprising a first process buffer comprised of 0.004 mL/mL of a 5% sodium azide solution, 0.014 mL/mL of 10N sodium hydroxide, and 0.0014 g/mL of lithium dodecyl sulfate. The swab was twirled in the first process buffer for 5 seconds to release stool from the swab. 30 μL of the buffer+sample was added to a second container containing a second process buffer. The second process buffer comprised 0.004 mL/mL of a 5% sodium azide solution, 0.0061 mL/mL of 5M NaCl, 0.0100 mL/mL of 1M Tris-HCl at a pH of 8.0, 0.002 mL/mL of 0.5M EDTA at a pH of 8.0, and 0.0000005 mL/mL of a control plasmid. The solution was mixed by pipeting 500 μL up and down 4-5 times.

It has also been observed that, in some assays (e.g., for C. difficile, HSV and VZV), no separate extraction step is necessary.

Separately, 135 μL of a rehydration solution was added to a solid reagent composition. 15 μL of the rehydrated reagent composition was added to each plate well. 5 μL of the diluted sample was added to each well. The plate was then placed into the Applied Biosystems® 7500 FastDx thermal cycling instrument.

The samples were also tested using the GeneOhm assay available from BD, which tests for the C. difficile toxin B gene (tcdB) only. The results of both assays are shown in Table 3.

TABLE 3
Platform Comparison with Clinical Specimens

| | BD GeneOhm | | |
|---|---|---|---|
| Present Test | | + | − |
| | + | 18 | 1 |
| | − | 0 | 64 |

Thus, the present assay had 100% positive agreement and 98.4% negative agreement with the BD GeneOhm test for Clostridium difficile from a stool specimen.

The limit of detection (LoD) for two strains of C. difficile was determined using quantified cultures of he strains serially diluted in negative specimen. 20 replicates were tested following the above assay workflow. The LoD was defined as the lowest concentration at which at least 95% of all replicates tested positive with the results shown in Table 4.

TABLE 4
Clostridium difficile Limit of Detection

| | | LOD |
|---|---|---|
| Strain | Toxinotype | CFU/assay |
| ATCC BAA-1870 | IIlb | 2.55E−01 |
| ATCC BAA-1872 | 0 | 9.50E−01 |

Testing against isolates or purified nucleic acids of 19 other viruses at clinically relevant levels confirmed that the reagents do not cross react with other common pathogens.

Testing against a panel of 21 C. difficile strains showed that the assay is inclusive of all the strains tested (Table 5).

TABLE 5
Clostridium difficile Assay Inclusivity

| C. difficile Strain | Toxinotype | CFU/assay | Result |
|---|---|---|---|
| ATCC BAA-1870 | IIIB | 8.50E−02 | Positive |
| CCUG 37770 | IV | 3.63E−01 | Positive |
| ATCC BAA-1803 | III | 1.64E−01 | Positive |
| CCUG 20309 | X | 2.83E+00 | Positive |
| CCUG 37774 | XXIII | 2.14E−01 | Positive |
| ATCC BAA-1872 | 0 | 9.50E−01 | Positive |
| ATCC BAA-1875 | V | 2.68E+00 | Positive |
| ATCC 43255 | 0 | 2.28E+00 | Positive |
| ATCC 43600 | 0 | 1.73E+00 | Positive |
| CCUG 9004 | NA | 1.58E+00 | Positive |
| CCUG 37773 | NA | 2.70E+01 | Positive |
| CCUG 37778 | NA | 3.24E+01 | Positive |
| ATCC 43599 | 0 | 1.41E+01 | Positive |
| CCUG 37777 | NA | 1.79E+01 | Positive |
| ATCC 700792 | NA | 9.75E+00 | Positive |
| ATCC 43598 | VIII | 9.44E+00 | Positive |
| ATCC 9689 | 0 | 1.32E+01 | Positive |
| ATCC 17858 | NA | 1.33E+01 | Positive |
| ATCC BAA-1805 | III | 1.07E+01 | Positive |
| ATCC BAA-1382 | NA | 8.66E+00 | Positive |
| CCUG 37776 | NA | 1.07E+01 | Positive |

Thus, the present assay was effective to broadly detect C. difficile strains including the hypervirulent strain NAP027.

The present assay was tested as described above with a panel of 19 bacteria and was found to be specific to toxigenic C. difficile targets. Specifically, the present test was not cross reactive with Candida albicans, Enterococcus faecalis, Escherichia coli, Escherichia coli O157:H7, Pseudomonas aeriginosa, Serratia marcesens, Staphylococcus aureus, Staphylococcus epidermidis, Streptococcus dysgalactiae (grp C and grp G), Streptococcus agalactiae (grp. B), Salmonella enteritidis, Shigella flexneri, Shigella sonnei, Clostridium difficile (nontoxigenic), Clostridium sordellii, Clostridium bifermentans, Clostridium perfringens, and Bacillus cereus.

A panel of 11 potentially interfering substances (Table 6) was evaluated at approximately 3×LOD and shown not to interfere with the present assay. The ATCC BAA-1870 (4.73E+01 CFU/mL) C. difficile strain was used in the interference assays.

TABLE 6
Potentially Interfering Substances

| Substance | Concentration | | Solvent | | Result |
|---|---|---|---|---|---|
| Palmitic Acid | 1.3 | mg/mL | 100% | methanol | Positive |
| Triclosan | 0.1% | (w/v) | 20% | DMSO | Positive |
| Triclosan | 0.1% | (w/v) | 100% | DMSO | Positive |
| Methicillin | 13 | mg/mL | water | | Positive |
| Phenylephrine HCl | 2% | w/v | water | | Positive |
| Phenylephrine HCl | — | | cream swab | | Positive |
| (cream) | | | | | |
| Stearic Acid | 26 | mg/mL | 100% | DMSO | Positive |
| Mineral Oil | 2% | v/v | 10% | DMSO | Positive |

| | | | | |
|---|---|---|---|---|
| Naproxen Sodium | 14 | mg/mL | water | Positive |
| Aluminum Hydroxide | 0.1 | mg/mL | water | Positive |
| Magnesium Hydroxide | 0.1 | mg/mL | water | Positive |
| Mucin | 3 | mg/mL | water | Positive |

### Example 2

### Multiplex Assays for Influenza A and B and for RSV A, RSV B, and hMPV

Multiplex real-time TaqMan® assays were developed to detect influenza A and B and RSV A, RSV B, and hMPV. RNA was extracted on either a NucliSENS® easyMag® or Roche MagNA Pure Compact, and 5 μl of each sample was added to reconstituted master mix. The Influenza assay master mix (solid composition) contained primers/probes for detection and differentiation of Influenza A and Influenza B, as shown in Table 1 above; the RSV/hMPV master mix contained primers/probes for the detection of RSV A, RSV B and hMPV. The assays followed the basic protocol set forth above.

Each cultured influenza A and B isolate was detected; 19/19 samples and 14/14 samples, respectively. Clinical specimens analyzed for the presence of either RSV A, RSV B or hMPV were able to detect 10/10 RSV A, 13/13 RSV B, and 26/26 hMPV. (Typing of RSV A vs. RSV B was done in a separate assay.)

Specificity was 100% for all samples evaluated. Initial analytical sensitivity tests for the various viruses indicated detection limits less than 50 TCID₅₀/ml and/or 10 vp/mL for each target. Testing with isolates of other common viruses and bacteria confirmed that these reagents are not cross reactive with other common respiratory pathogens.

### Example 3

### Multiplex Assay for HSV-1, HSV-2 and VZV

A multiplex real-time TaqMan Assay® was developed to detect and differentiate HSV-1, HSV-2 and VZV. The assay master mix (solid composition) contained primers/probes for detection and differentiation of these three analytes. The assay followed the basic protocol set forth above. In some instances, however, no extraction step is required. Testing was performed on cultured isolates of viruses to establish the initial performance characteristics of the assay. Initial LoD studies with the three viruses showed detection limits of less than 20 copies/assay on the Applied Biosystems® 7500 FastDx platform. Initial clinical performance of the test was carried out with previously characterized frozen specimens.

Results showed that 10/10 specimens tested HSV-1 positive, 9/9 specimens tested HSV-2 positive, 11/11 specimens tested VZV positive, and 4/4 negative specimens tested negative, in concordance with previous testing results. Testing against isolates or purified nucleic acids of 19 other viruses at clinically relevant levels confirmed that the reagents do not cross react with other common pathogens.

These and other applications and implementations will be apparent in view of the disclosure. Such modifications, permutations, additions, substitutions, alternatives and sub-combinations thereof can be made without departing from the spirit and scope of the invention, which should be determined from the appended claims. While the present device, system, and method have been described with reference to several embodiments and uses, and several drawings, it will be appreciated that features and variations illustrated or described with respect to different embodiments, uses, and drawings can be combined in a single embodiment.

What is claimed is:

1. A method for identifying the presence or absence of a Clostridium species in a sample, the method comprising:
a) spiking a sample suspected of containing a target nucleic acid sequence of a Clostridium species with a process control sequence, to form a spiked solution;
b) exposing the spiked solution to lysing conditions to form a lysed solution;
c) contacting said lysed solution with an amplification solution to form a mixture, the amplification solution comprising
(i) at least two PCR analyte primer pairs, each pair specific for a different target nucleic acid sequence of a Clostridium species;
(ii) a PCR control primer pair specific for said process control sequence;
(iii) at least one analyte probe specific for each said different target nucleic acid sequence;
(iv) a control probe specific for said process control sequence;
(v) a thermostable enzyme having DNA polymerase activity; and
(vi) deoxyribonucleotides dATP, dCTP, dGTP and dTTP or dUTP;
d) producing an amplicon from at least one target nucleic acid sequence in the mixture, if present, using a single set of thermocycling conditions in a thermocycler; and
e) monitoring the at least one analyte probe to determine the presence or absence of the target nucleic acid sequence.

2. The method of claim 1, further comprising:
prior to spiking the sample, processing the sample comprising
(a) adding the sample to a first processing buffer to produce a buffered sample; and
(b) adding a portion of the buffered sample to a second processing buffer.

3. The method of claim 1, wherein the melting temperatures (Tm) of primer/binding site duplexes for the different analytes and for the process control are within 3° C. in the PCR reaction environment.

4. The method of claim 1, wherein the amplification solution comprises manganese acetate.

5. The method of claim 1, wherein the at least two PCR analyte primer pairs are each specific for a different target nucleic acid sequence originating from Clostridium difficile.

6. The method of claim 5, wherein the different target nucleic acid sequences are transcribed from RNA of tcdA and tcdB genes of Clostridium difficile.

7. The method of claim 1, wherein the sample is a stool sample.

8. A composition, comprising:
at least two PCR analyte primer pairs, each pair substantially complementary to a different target DNA sequence derived from Clostridium difficile;
a PCR control primer pair substantially complementary to a process control DNA sequence;
at least one analyte probe for specific binding to each said target DNA sequence;
a control probe for binding to the process control DNA sequence;
a thermostable enzyme having DNA polymerase activity; and
deoxyribonucleotides dATP, dCTP, dGTP and dTTP or dUTP;
wherein said composition is in solid form,
and wherein said analyte primer pairs and control primer pair are designed such that amplification and detection of said different target DNA sequences and said process control DNA sequence can be performed simultaneously using the same thermal cycling conditions on a thermocycler.

9. The composition of claim 8, wherein the melting temperatures (Tm) of primer/binding site duplexes for the different analytes and for the process control are within 3° C. in the PCR reaction environment.

10. The composition of claim 8, wherein the different target DNA sequences are transcribed from RNA of tcdA and tcdB genes of Clostridium difficile, respectively.

11. A kit comprising:
a first container containing a composition according to claim 8; and
a second container containing a rehydration solution.

12. The kit of claim 11, wherein the rehydration solution comprises manganese acetate.

13. The kit of claim 11, further comprising a third container containing a solution of MS-2 phage.

14. The kit of claim 11 further comprising a fourth container containing a first process buffer, and a fifth container containing a second process buffer.
